(19) United States
(12) Patent Application Publication
(10) Pub. No.: US 2002/0042562 A1
(43) Pub. Date: Apr. 11, 2002

(54) Immobilizable in vivo sensing device

(76) Inventors: Gavriel Meron, Petach Tikva (IL); Mark G. Gilreath, Charlotte, NC (US); Yoram Ashery, Givat Shmuel (IL); Reuven Shreiber, Haifa (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 09/963,950

(22) Filed: Sep. 26, 2001

Related U.S. Application Data
(60) Provisional application No. 60/235,583, filed on Sep. 27, 2000.
(60) Provisional application No. 60/311,351, filed on Aug. 13, 2001.

(51) Int. Cl. A61B 5/00

(52) U.S. Cl. 600/361; 600/476; 600/547; 600/549; 128/903

(57) ABSTRACT

A system for monitoring a site in vivo comprising a housing configured for being immobilized in vivo; at least one sensing device for obtaining data of the site, the sensing device connected to the housing; a transmitter for transmitting the data obtained by the sensing device;, and a receiving system for receiving the data transmitted by the sensor

### FIELD OF THE INVENTION

The present invention relates to in vivo sensing devices. More specifically, the present invention relates to a system and method for immobilizing a sensing device in vivo, for example, for post surgery monitoring.

### BACKGROUND OF THE INVENTION

In vivo sensing devices, such as thermometers, pH meters, optical scanners, image sensors and so on, can be used for unobtrusively monitoring bodily systems. Some in vivo sensors move through body lumens and can be remotely controlled. However, it is sometimes desirable to immobilize a sensing device in vivo for continuous sensing of an in vivo site, for example for post surgery monitoring.

In the time immediately after surgery patients frequently experience organ functional problems. For example, during surgery in the gastrointestinal tract the blood pressure at the vicinity of the surgical site is reduced and peristalsis is arrested. After surgery the blood pressure increases and peristalsis is resumed sometimes causing bleeding from the surgical site into the intestine lumen.

Also, for example, in treating coronary artery disease, it is sometimes necessary to bypass coronary arteries with a vascular graft, which is surgically attached to the heart, to circumvent a blocked coronary artery. After surgery cardiac functional problems may occur due to build-up of stenotic lesions or other obstructions to the flow of blood through the implanted graft.

Postoperative monitoring of the gastrointestinal tract is important to avoid letting too much time elapse before blood loss into the intestine is detected.

Similarly, it is important that the condition of a vascular graft be monitored, post-surgery, to detect the further build-up of stenotic lesions or other obstructions to the flow of blood through the implanted is graft.

Various catheterization procedures are known for assessing the flow characteristics of a blood vessel or blood vessel graft. However, the introduction of catheters into the vascular system may result in damage to blood vessels.

U.S. Pat. No. 4,915,113 to Holman describes an implantable system for monitoring blood flow through surgically implanted grafts The system, which comprises Doppler crystal transducers, utilizes a subcutaneously implanted electrical plug-type connector, accessible through an incision at the implant site, and electrical conductors to connect terminals on that plug to the Doppler crystal transducers.

Ultrasound echo imaging is known for visualization and examination of a patients heart. However, methods of echocardiography do not always result in good quality images after cardiac surgery.

Monitoring of in vivo processes, not necessarily related to post surgical events, may also be an important diagnostic tool. For example, in endometriosis, in which cells that normally grow inside the uterus instead grow outside the uterus. Endometrial cells line the uterus and are normally shed each month during menstruation. When endometrial cells grow outside the uterus, the cells implant. These implants occur most commonly within the fallopian tubes and on the outside of the tubes and ovaries, the outer surface of the uterus and intestines and anywhere on the surface of the pelvic cavity. They can also be found, less often, on the surface of the liver, in old surgery scars or, very rarely, in the lung or brain. The implants cause internal bleeding, which leads to tissue inflammation and later, scarring and possibly infertility. Endometriosis can be suspected based on symptoms of pelvic pain and findings during physical examinations in the doctor's office but neither the symptoms nor the physical examination can be relied upon to establish the diagnosis of endometriosis. Imaging studies, such as ultrasound, can be helpful in studying the pelvis, but still cannot accurately diagnose endometriosis. Direct visual inspection and tissue biopsy of the implants are necessary for accurate diagnosis. Currently, the only accurate way of diagnosing endometriosis is at the time of surgery (either by open standard laparotomy or laparoscopy).

### SUMMARY OF THE INVENTION

The present invention provides a system and method for immobilizing a sensing device in vivo. In the present invention, data relating to environmental conditions at an in vivo site, or images of an in vivo site, are obtained over a specific period and thus the invention enables monitoring of an in vivo site. For example, the invention enables post surgery monitoring of surgical operations, which provides camera or video images of the surgery site during the critical post surgery hours. The system and method of the invention enable an external operator to directly observe changes occurring at an in vivo site, such that pathological occurrences can be detected at their onset and immediately be treated.

The invention further provides an immobilizable in vivo sensing device that can be used for monitoring an in vivo site by utilizing any appropriate sensing device (pH meter, blood detector, imaging device etc.). In the gastrointestinal (GI) tract the device of the invention is immobilized for monitoring a site in the GI tract with out having to leave an opening in the patient or incise the patient twice for retrieval of the in vivo device at the end of the monitoring period.

The system of the invention comprises a housing configured for being immobilized in vivo, at least one sensing device connected to the housing and a reception system for receiving output from the sensing device. Preferably, the housing is configured for being transiently immobilized in vivo. In an embodiment of the invention the housing comprises means for anchoring the housing in a body lumen.

The sensing device, which may be a known in vivo sensing device, such as an in vivo pH meter, a thermometer, an optical scanner, an imaging device etc., may be contained in the housing or otherwise connected to the housing. Additional functional units of the sensing device, such as a transmitter for wirelessly transmitting data to an external receiving unit, an illumination source, a power source etc, may be part of the sensing device, or possibly may be contained in the housing. The housing and/or sensing device can be connected by wires to external sources. However, preferably, the sensing device and its functional units are wirelessly operable.

In one embodiment of the invention, the system comprises a housing having an optical window, said housing configured for being immobilized in the vicinity of an in vivo site, such as a surgical site; at least one imaging device, such as a charged couple device (CCD) or complementary metal oxide semiconductor (CMOS) image sensor contained within the housing; a transmitter that transmits the output of the imaging device; and a reception system for receiving the transmitted output The method of the invention comprises the steps of immobilizing, preferably transiently, a sensing device in the vicinity of an in Vivo site; sensing the in vivo site; and receiving sensed data of the in vivo site, externally to the surgical site.

In one embodiment the method comprises the steps of transiently immobilizing an imaging device in the vicinity of a site in the GI tract; imaging the in site; transmitting images of the site and receiving the images of the site externally to the GI tract.

There is thus provided, according to an embodiment of the invention, a method for post surgical monitoring of a surgical site in vivo.

### BRIEF DESCRIPTION OF THE FIGURES

The present invention will be understood and appreciated more fully from the following detailed description taken in conjunction with the figures in which:

FIG. 1 is a schematic illustration of some components of the system in accordance with an embodiment of the invention;

FIG. 2 is a tangential section schematic illustration of some components of the system in accordance with another embodiment of the invention;

FIG. 3A and 3B are schematic illustrations of an in vivo sensing device according to further embodiments of the invention; and FIG. 4 is a schematic illustration of some components of the system in accordance with yet another embodiment of the invention

DETAILED DESCRIPTION OF THE INVENTION

The present invention provides a system and method for monitoring an in vivo site, which provide information related to the in vivo site for the duration of the monitoring period.

The system typically includes a sensing device, a transmitter that transmits the output of the sensing device, a reception system for receiving the transmitted output and a power source, which provides power to the elements of the system. At least the sensing device is connected to a housing, which is configured for being transiently immobilized in the vicinity of the surgical site (further described in the Figures). The sensing device may be any device that is adapted for being placed in vivo (for example, along the GI tract) that can sense environment conditions such as the presence of blood, pH, temperature, electrical impedance of tissues etc., and that can transmit (such as by radio) output relating to changes in the environment conditions.

The invention can be utilized for monitoring in vivo sites in diverse body systems, as will be exemplified below. In one embodiment there is provided a system for monitoring site in the GI tract, in which the sensing device is an imaging system. The imaging system typically includes at least one illumination source such as a white LED (light emitting diode) and an imaging device such as a CCD or CMOS image sensor. The imaging system may further include an optical system for imaging an area of interest onto the imaging system. The optical system may comprise mirrors and/or lenses for collimating the light from the illumination source. In accordance with this embodiment the reception system receives the transmitted video output and may include a) an antenna array capable of surrounding a body for receiving the transmitted video output and for producing a plurality of received signals and b) a demodulator capable of transforming the plurality of received video signals into a single video data stream Optionally the reception system includes a display, such as LCD, for displaying the data transmitted to it For example, a system, which includes a camera system, a transmitter and a receiving system such as described in U.S. Pat. No. 5,604,531 or in U.S. patent application Ser. No. 09/800,470, may be used in the present invention U.S. Pat. No. 5,604,531, and U.S. patent application Ser. No. 09/800,470 are assigned to the common assignee of the present invention and are hereby incorporated by reference.

The imaging system provides direct visual information of the in vivo site (i.e., a surgical site) such that visibly detectable changes at the site, such as bleeding, swelling etc. can be seen by an external operator. The imaging system may further comprise a detector coupled to the imaging device that is optically changed in response to changes in environmental conditions. The optical change in the detector is imaged and transmitted to a receiving system and shown on a display of the receiving unit to alert an external operator of the changed conditions. For example, the imaging system may comprise a pH meter that undergoes a color change in response to pH changes in its vicinity. Also, the imaging system may comprise a detector of chemical substances, such as blood components, which undergoes a change in color in response to the presence of the chemical substances. In both cases a change in color will be detected by the imaging device and will be transmitted and received by the reception system for the inspection of an external operator.

The sensing device, such as an imaging system, may further be in communication with a processor/control for analyzing the data detected by it and possibly for controlling the sensing device. For example, images of a surgical site may be transmitted to a processor where they are analyzed for the presence and possibly the concentration of blood (by detecting certain changes in color). The image may then be received by the external operator including additional information, generated by the processor, regarding the bleeding at the surgical site. Further, the system may include means for alerting the external operator. The means for alerting the external operator are in communication with the processor. Thus, when the presence of blood is detected by the processor a signal, such as a flashing light or an alarm, may be activated to alert the external operator.

The housing, according to the invention, is configured for being immobilized in the vicinity of an in vivo site. The housing in some exemplary embodiments of the invention is schematically illustrated in FIGS. 14.

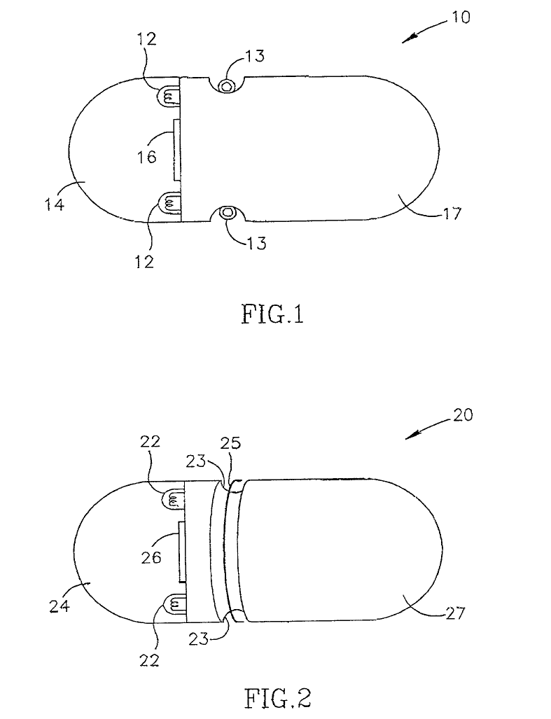

The housing illustrated in FIG. 1 is a capsule 10 designed to passively transverse the GI tract. Capsule 10 comprises a capsule body 17, which includes an optical window 14 behind which are positioned illumination sources 12 and an imaging device 16. The capsule body 17 houses additional elements of the system (not shown), such as a processor/controller for processing image data obtained by the imaging device 16 and possibly for controlling imaging device 16, a transmitter for transmitting images of the surgical site to an external reception system and a power source, such as a battery. Capsule 10 has two rings 13 on its perimeter, about equally distanced from each other. The rings 13 are fit into depressions in the capsule body 17 perimeter so that they do not protrude from the capsule 10 perimeter and do not obstruct the capsules passage through the GI tract. Rings 13 are used for sewing the capsule 10 to a desired location in vivo, for example in the vicinity of a surgical site in the GI tract. Following a surgical operation in the GI tract the surgeon places capsule 10 at a location in the vicinity of the surgical site with the optical window 14 facing the surgical site. The surgeon then immobilizes the capsule to its place by sewing a suture through ring 13 and through the issue at the location. The capsule 10 is thus fastened to the tissue from two sides of the capsule 10. It should be appreciated that one or more rings can be used in the invention, depending, inter alia, on the shape of the housing and the anatomy of the in vivo site,

The imaging device 16, which is typically activated when the capsule 10 is immobilized or just prior to that, can be preprogrammed to image the surgical site at any desired rate. Optionally, the rate of obtaining images can be controlled either by an external operator or by a processing/controlling unit, as described above, wherein detection of blood, for example, will cause an increased rate of image acquisition Typically, the components of the system, such as the imaging device 16, the illumination sources 12 and the transmitter are battery powered. The life of the battery depends mostly on the rate of imaging. In most cases, the rate of imaging required is low (typically one image every few hours) enabling a battery to sustain the operation of the system for a period of days or even weeks.

The sutures used to immobilize capsule 10 may be degradable, as further described below, such that the capsule 10 can be freed from its location after a period of monitoring and can then be removed by a surgeon, or, in the case of the GI tract, the capsule 10 will proceed to passively move through the GI tract and be naturally excreted from the body.

FIG. 2 schematically illustrates a tangential section of capsule 20, which similarly to capsule 10, comprises a capsule body 27 having an optical window 24 behind which are positioned illumination sources 22 and an imaging device 26. Other elements of the system, such as a processor/controller, a transmitter and power source are housed in the capsule body 27. Capsule 20 is ellipsoid shaped having an indentation 23 circling the entire capsule 20 perimeter more or less around the center of the capsule 20. Indentation 23 forms a groove suitable for accommodating the operating doctor's thread 25. The capsule 20 is thus fastened to the surgical site by thread 25 which surrounds the capsule 20 and which is anchored into the patients body Typically, the thread used for suturing the capsule 10 or 20 to a surgical site in vivo is thread, which will disintegrate with time. Thus, a doctor performing an operation in the gastrointestinal tract can activate the system (initiate imaging) in capsule 10 or 20 and sew in the capsule 10 or 20 at the operation site in the gastrointestinal tract prior to closing the surgical incision. The imaging, which may be continues or periodical, lasts through the critical post surgical period (up to 24 hours after the operation). During this time the surgical site will be imaged and the images will be transmitted to the receiving system, such as an external workstation, where the images will be monitored by an external operator. At some point in time, either during the imaging process or after its termination, the sutures sewn through rings 13, or around capsule 20 in indentation 23, which have been immobilizing the capsule to the surgical site, will disintegrate and capsule 10 or 20 will be released into the gastrointestinal tract. The capsule 10 or 20 will be free to travel through the GI tract driven by peristalsis and will be naturally excreted from the body.

The system and method of the invention thus enable post surgical monitoring in the gastrointestinal tract with out having to leave an opening in the patients body or having to cut the patient a second time in order to retrieve the monitoring system.

In other embodiments a housing comprising an imaging system, as described above, may be designed for other surgical sites than the GI tract, such as the lungs or blood vessels. In these embodiments a housing, which comprises an optical window, may comprise a clip or any other means for immobilizing the housing to a site of interest at the time of surgery and for being later removed, for example, through an incision or through a transthoracic or transesophageal opening.

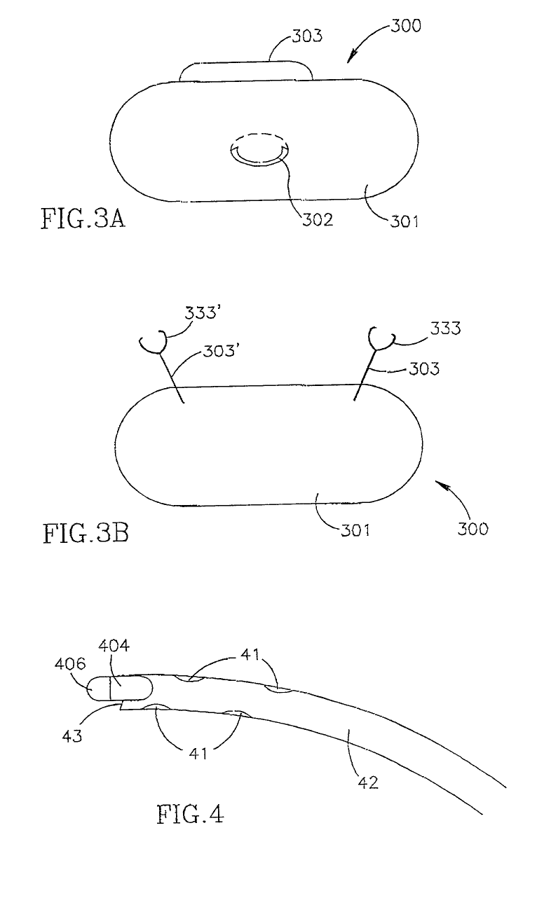

Reference is now made to FIGS. 3A and 3B, which are schematic illustrations of an in vivo sensing device according to further embodiments of the invention. In FIG. 3A an in vivo sensing device 300 comprises a housing 301 connected to a sensor 303. The housing 301 may also be connected to a transmitter for transmitting the data obtained by the sensor 303 and optionally an internal power source, such as a battery or an externally inducible power source. The housing 301 includes a niche 302 configured for receiving means for anchoring the device 300 to an internal body tissue. The anchoring means may include a suture or other sewing means, wherein a stitch is sewn through niche 302 and through an internal body tissue. Optionally the suture may be biodegradable. In another embodiment the suture may be in the form of a knot made of a shape memory alloy or a shape memory polymer, which are able to change shape in response to temperature, as known in the art. The shape memory material knot could be induced to change its shape either to tighten the knot thus immobilizing the device 300 to a proximal tissue or to loosen the knot, thus freeing the device 300 from a proximal tissue. This change of shape could be induced, for example, by the temperature prevalent in the body lumen or by heating or cooling through electrical means, for example electrical means generated by a battery in the device 300.

In FIG. 3B the in vivo sensing device 300 comprises a housing 301 which includes clasps 303 and 303′. Clasps 303 and is 303′, which typically extend from the housing 301, include fasteners 333 and 333′ for releasably grasping an internal body tissue. The fasteners 333 and 333′ may hold on to an internal body tissue by digging into the tissue, through suction or by any other suitable means. Any suitable number and type of clasps and/or fasteners may be used.

The device 300 typically stores in the housing 301 a sensor or a plurality of sensors (not shown), such as a pH meter, a pressure detector, a temperature sensor or an optical sensor, such as an image sensor, or any combination of sensors. Alternatively any suitable sensor could be attached externally to housing 301 Device 300 may be delivered to an in vivo site as described in the art, for example, by an endoscope into the GI tract At the in vivo site the device 300 can be immobilized for a predetermined time to an internal body tissue, such as to parts of the GI tract or to the inside of blood vessels etc. Device 300, thus immobilized, can be used for sensing an in vivo site over a predetermined period. The device 300 can also be utilized for simultaneously sensing an in vivo site and performing an in vivo procedure such as diagnostic and/or therapeutic procedures performed in the human body, for example, but not limited to, procedures of gastroenterology, procedures within blood vessels, procedures of gynecology and laparoscopic surgery procedures. The in vivo procedures can be performed by specialized arms protruding from the device, such as arms described in U.S. Pat. No. 6,240,312 to Alfano.

In one embodiment of the invention the housing is connected to an imaging unit, The imaging unit includes an illumination source, such as light emitting diodes (LEDs), an image sensor, such as a CCD or CMOS image sensor and an optical system for imaging an in vivo site onto the image sensor. The housing is connected also to a transmitter for wirelessly transmitting image data to an external receiving system In this embodiment the housing also includes a battery for powering all the electrical elements of the imaging system and the transmitter. The imaging unit and transmitter will be referred to as an imaging system. The imaging system according to this embodiment can operate in a manner similar to the imaging systems that are described in the above mentioned U.S. Pat. No 5,604,531 or in U.S. patent application Ser. No. 09/800,470.

In an optional embodiment of the invention the sensing device includes an image sensor and the clasps include grasping means and fastening means for initially grasping an internal tissue and then fastening the device to the tissue. This embodiment could be similar to the known pH sensor having suction means and pins for grasping by suction the internal tissue in the esophagus and for fastening the grasped tissue to the pH sensor by the pins.

Reference is made to FIG. 4 in which a housing 404 having an optical window 406, is attached to the distal end 43 of a drain catheter 42. The housing 42 comprises an imaging system capable of imaging an in vivo site through optical window 406.

Drain catheter 42 comprises lateral openings 41 such that drainage can be carried out through openings 41. Drain catheter 42 is inserted into a patients body for draining an in vivo site, such as an abscess or at a site of surgery. The distal end 43 of the drain catheter 42 is inserted into a patient's body while the other end is left out side of the patient's body. Liquids and debris passing into the drain catheter 42 through openings 41 and possibly through an opening at the distal end 43, can thus be drained and removed from the body. Drain catheter 42 is inserted into a patients body through surgery or by minimal invasive methods, for example, by using ultrasound or CT for guiding the catheter's distal end 43 to the required site in vivo.

Housing 404, which is attached to the distal end 43 of the drain catheter 42 is immobilized to the site of drainage enabling the imaging system it comprises to obtain images through optical window 406. Thus, an imaging system can be brought to and held at the drainage site for monitoring the drainage process. As described above, the rate of imaging can be predetermined or controlled by an external operator or controlled automatically in response to conditions detected in the images obtained of the site Monitoring a site of drainage in accordance with this embodiment of the invention enables an external operator to easily see, without using eternal techniques such as CT, if the site has been drained, to see that there is no active bleeding at the site, etc., or if there is leaking, to identify the site of leaking.

Further, in accordance with an embodiment of the invention an imaging system as described above can be immobilized in vivo, according to the invention, for monitoring in vivo processes. For example, an immobilizable imaging system according to an embodiment of the invention can be immobilized in a uterus for monitoring the development of a fetus in that uterus. The imaging system, which is battery powered and which wirelessly transmits images to an external recording system, can be programmed to obtain images of the fetus in predetermined intervals, such as once every 24 hours. Consecutive, though not necessarily continuous, images of the fetus will provide means for visually monitoring the development of a fetus and, at the same time, will ensure a long life of the battery so as to enable imaging over a long period (i.e., the term of pregnancy). Alternatively, the imaging system may be externally induced, as known in the art. In addition to continuous monitoring of the development of the fetus, real time images can be obtained when required, for example, when the patient is visiting the doctor or when the patient is experiencing difficulties and visual site of the fetus may provide an explanation for the experienced difficulties.

An immobilizable imaging system according to an embodiment of the invention may also be used for diagnosing and/or monitoring in vivo procedures such as endometriosis. An immobilizable imaging system according to an embodiment of the invention can be immobilized at sites of endometrial implants or of suspected implants, such as within the fallopian tubes, and transmit images of the site to an external receiving system for detecting or monitoring endometrial implants.

Also, an immobilizable imaging system according to an embodiment of the invention can be immobilized in a blood vessel, for example, for monitoring restinosis after implantation of a stent. The imaging system can be immobilized at the site of the stent implantation and images of the site can be obtained, as above, at predetermined intervals, for example, once a week. Consecutive, though not necessarily continuous images of the site of the stent implantation, will provide means for warning a physician of the occurrence of restinosis or any other pathologies related to the stent.

In another embodiment of the invention the housing comprises an imaging system, for example, as described above, and further comprises a detector of substances in an in vivo environment, such as blood, sugar, amino acids, microorganisms etc, or of conditions prevalent in an in vivo environment, such as pH, temperature etc. The detector, which is adhered to the housing in such a way that it is included in the angle of view of the imaging system, reacts to the presence of substances or environmental conditions by causing an optical change. An example of such a detector may be a strip of pH sensitive material that is adhered to the optical window of the imaging system. Other examples are described in WO 01/53792, which is assigned to the common assignee of the present invention and which is hereby incorporated by reference. A device according to this embodiment, which is immobilized at an in vivo site, can provide an external operator with images of the in vivo site and simultaneously with information relating to the environmental conditions at the in vivo site.

It will be appreciated by persons skilled in the art that the present invention is not limited by what has been particularly shown and described herein above. Rather the scope of the invention is defined by the claims which follow:

1. A system for monitoring a site in vivo comprising
a housing configured for being immobilized in vivo;
at least one sensing device for obtaining data of the site, said sensing device connected to said housing;
a transmitter for transmitting the data obtained by the sensing device; and
a receiving system for receiving the data transmitted by the sensor.

2. The system according to claim 1 further comprising a processing unit for processing the data obtained from the sensing device.

3. the system according to claim 1 further comprising a processing/controlling unit for processing the data obtained from the sensing device and for controlling the sensing device in accordance with the data obtained from the sensing device.

4. The system according to claim 1 wherein the receiving system comprises a display for displaying the transmitted data.

5. The system according to claim 1 further comprising an internal power source.

6. The system according to claim 5 wherein the internal power source is a battery.

7. The system according to claim 1 wherein the sensing device is selected from the group consisting of: an optical scanner, a pH meter, a thermometer, a sensor of electrical conductivity of tissues or an image sensor.

8. The system according to claim 1 wherein the sensing device is a pH meter.

9. The system according to claim 1 wherein the sensing device is an image sensor.

10. The system according to claim 9 wherein the image sensor comprises a detector that is optically changed in response to changes in environmental conditions

11. The system according to claim 1 wherein the transmitter is a wireless transmitter.

12. The system according to claim 1 wherein the housing is configured for being sewn to an in vivo site.

13. The system according to claim 1 wherein the housing comprises at least one ring on the perimeter of the housing for threading a suture there through.

14. The system according to claim 1 wherein the housing comprises an indentation around the perimeter of the housing, said indentation configured for receiving a suture.

15. The system according to claim 1 wherein the housing comprises a niche configured for receiving means for anchoring the housing to a body tissue.

16. The system according to claim 15 wherein the means for anchoring the housing to a body tissue are selected from the group consisting of: pins, clasps, fasteners and suction means.

17. The system according to claim 1 wherein the housing comprises means for anchoring the housing to a body tissue.

18. The system according to claim 17 wherein the means for anchoring the housing to a body tissue are selected from the group consisting of: pins, clasps, fasteners and suction means.

19. A system for post surgery monitoring comprising
a housing configured for being immobilized in the vicinity of a surgery site in vivo;
at least one sensing device for obtaining data of the site, said sensing device connected to said housing;
a transmitter for transmitting the data obtained by the sensing device; and
a receiving system for receiving the data transmitted by the sensor.

20. The system according to claim 19 wherein the sensing device is an image sensor.

21. The system according to claim 1 for monitoring a site in the GI tract.

22. The system according to claim 21 for monitoring a site in GI tract.

23. An immobilizable in vivo sensing device comprising a housing configured for being immobilized in vivo and an in vivo sensor for obtaining in vivo data.

24. The device according to claim 23 further comprising a processing unit for processing the data obtained from the in vivo sensor.

25. The device according to claim 23 further comprising a processing/controlling unit for processing the data obtained from the in vivo sensor device and for controlling the device in accordance with the data obtained from the in vivo sensor.

26. The device according to claim 23 further comprising an internal power source.

27. The device according to claim 26 wherein the internal power source is a battery.

28. The device according to claim 23 wherein the in vivo sensor is selected from the group consisting of an optical scanner, a pH meter, a thermometer, a sensor of electrical conductivity of tissues or an image sensor.

29. The device according to claim 23 wherein the sensing device is a pH meter.

30. The device according to claim 23 wherein the sensing device is an image sensor.

31. The device according to claim 23 wherein the image sensor comprises a detector that is optically changed in response to changes in environmental conditions.

32. The device according to claim 23 further comprising a transmitter for transmitting the data obtained by the in vivo sensor.

33. The device according to claim 32 wherein the transmitter is a wireless transmitter.

34. The device according to claim 23 wherein the housing is configured for being sewn to an in vivo site.

35. The device according to claim 23 wherein the housing comprises at least one ring on the perimeter of the housing for threading a suture there through.

36. The device according to claim 23 wherein the housing comprises an indentation around the perimeter of the housing, said indentation configured for receiving a suture.

37. The device according to claim 23 wherein the housing comprises a niche configured for receiving means for anchoring the housing to a body tissue.

38. The device according to claim 23 wherein the means for anchoring the housing to a body tissue are selected from the group consisting of: pins, clasps, fasteners and suction means.

39. The device according to claim 23 wherein the housing comprises means for anchoring the housing to a body tissue.

40. The device according to claim 39 wherein the means for anchoring the housing to a body tissue are selected from the group consisting of: pins, clasps, fasteners and suction means.

41. A drain catheter having a distal end and a proximal ends wherein the distal end is inserted into a patient's body and wherein the proximal end is out side of the patient's body, said catheter comprising an imaging device attached to the distal end of the catheter for obtaining in vivo data.

42. The catheter according to claim 41 wherein the imaging device comprises an at least one image sensor and at least one illumination source.

43. The catheter according to claim 42 further comprising a battery.

44. The catheter according to claim 41 wherein the imaging device comprises a detector that is optically changed in response to changes in environmental conditions.

45. The catheter according to claim 41 further comprising a transmitter for transmitting the in vivo data.

46. The catheter according to claim 45 wherein the transmitter is a wireless transmitter.

47. A method for monitoring an in vivo site comprising the steps of
Immobilizing a sensing device in the vicinity of an in vivo site;
sensing the in vivo site; and
receiving sensed data of the in vivo site.

48. The method according to claim 47 further comprising the step of transmitting sensed data prior to the step of receiving the second data.

49. The method according to claim 47 wherein the sensing device is an imaging device.

50. The method according to claim 47 wherein receiving the sensed data is done externally.

51. The method according to claim 47 wherein the in vivo site is in the GI tract.
